(19) United States
(12) Patent Application Publication
(10) Pub. No.: US 2005/0182089 A1
(43) Pub. Date: Aug. 18, 2005

(54) Pharmaceutical composition comprising a monoamine neurotransmitter re-uptake inhibitor and an N-methyl-D-aspartate (NMDA) receptors antagonist

(75) Inventors: Thomas Friedl, Ochsenhausen (DE); Joachim Mierau, Mainz (DE); Andreas Raschig, Biberach (DE); Juergen Reess, Ulm (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 11/039,990

(22) Filed: Jan. 21, 2005

(30) Foreign Application Priority Data
Jan. 22, 2004 (EP) 04 001 283
Mar. 11, 2004 (EP) 04 005 818

(51) Int. Cl. A61K 31/46; A61K 31/195; A61K 31/13

(52) U.S. Cl. 514/304; 514/561; 514/651; 514/662

(57) ABSTRACT

Accordingly, the invention relates to a pharmaceutical composition comprising a monoamine neurotransmitter re-uptake inhibitor comprising a 2,3-disubstituted tropane moiety, or a tautomer, a pharmaceutically acceptable salt, solvate, or physiologically functional derivative thereof, and at least one NMDA receptor antagonists or a pharmaceutically acceptable salt, solvate, or physiologically functional derivative thereof.

This applications claims priority of EP Application Nos. 04001283 and 04005818, which are incorporated herein by reference in their entirties.

### BACKGROUND OF THE INVENTION

1. Technical Field

The present invention relates to a combination of a monoamine neurotransmitter re-uptake inhibitor and an NMDA receptors antagonist, and the use of the combination in treating neurodegenerative conditions such as dementia of Alzheimer type, cerebrovascular disease, and depression.

2. Background Information

Alzheimer's Disease and dementia of Alzheimer-type are insufficiently understood neurodegenerative conditions mainly affecting the elderly, but also younger people who are mainly genetically predisposed to it.

One postulated method of treatment comprises the administration of antagonists of NMDA receptors.

The tropane derivative having dopamine reuptake inhibitor activity for use according to the invention may, in particular, be tropane derivatives such as those disclosed by patent applications EP 604355, EP 604352, U.S. Pat. No. 5,444,070, EP 604354, WO 95/28401, and WO 97/30997, all of which are encorporated herein in their entirties.

The International patent application WO 97/30997 discloses tropane derivatives, which are monoamine neurotransmitter re-uptake inhibitors. Similar compounds are known from the International patent application WO 93/09814.

However, there is no hint to combine these compounds with an NMDA receptor antagonists.

The present invention provides a new and surprisingly effective combination of an NMDA receptor antagonist and a monoamine neurotransmitter re-uptake inhibitor for separate, sequential, or simultaneous administration.

Surprisingly the combination provides:
i) lower doses to be used as expected for the single drugs, and
ii) a reduction or minimization of the adverse event profile of each single drug which increases general tolerability and compliance of both substances and decrease any adverse side effects as the profile of each substance is totally different due to the different mechanism of action.

### BRIEF SUMMARY OF THE INVENTION

Accordingly, the invention relates to a pharmaceutical composition comprising a monoamine neurotransmitter re-uptake inhibitor comprising a 2,3-disubstituted tropane moiety, or a tautomer, a pharmaceutically acceptable salt, solvate, or physiologically functional derivative thereof, and at least one NMDA receptor antagonists or a pharmaceutically acceptable salt, solvate, or physiologically functional derivative thereof.

The present invention provides a greater than expected improvement than would be expected from administration of the active ingredients alone in the condition of subjects suffering from a neurodegenerative disorder with an associated cognitive deficit, such as Alzheimer's Disease, dementia of Alzheimer-type, Lewy Body dementia, fronto-temporal dementia, or from a cognitive deficit that may arise from a normal process, such as aging-like cerebrovascular dementia, multi-infarct dementia, and milder forms, such as age-associated memory impairment (AAMI) or mild cognitive impairment (MCI), or from an abnormal process, such as injury, than would be expected from administration of the active ingredients alone. Further, the combination allows for a lower overall dose of each of the active ingredients to be administered, thus reducing side effects and decreasing any reduction in the effectiveness of each of the active ingredients over time.

There is also provided a kit of parts comprising at least two separate unit dosage forms (A) and (B):
(A) comprising a composition a monoamine neurotransmitter re-uptake inhibitor comprising a 2,3-disubstituted tropane moiety, or a tautomer, a pharmaceutically acceptable salt, solvate, or physiologically functional derivative thereof (I), and optionally a pharmaceutically acceptable carrier;
(B) comprising a composition containing one or more NMDA receptor antagonists or a pharmaceutically acceptable salt, solvate, or physiologically functional derivative thereof, and optionally a pharmaceutically acceptable carrier,
   for simultaneous, sequential or separate administration.

There is also provided the use of a combination of a monoamine neurotransmitter re-uptake inhibitor comprising a 2,3-disubstituted tropane moiety, or a tautomer, a pharmaceutically acceptable salt, solvate, or physiologically functional derivative thereof (I), and at least one NMDA receptor antagonist, or a pharmaceutically acceptable salt, solvate, or physiologically functional derivative thereof in a combined form, or separately, or separately and sequentially, wherein the sequential administration is close in time or remote in time, for the prevention or treatment of a disease or a disorder, which is responsive to the inhibition of monoamine neurotransmitter re-uptake and/or to NMDA inhibition.

There is also disclosed a method of prevention or treatment of a disease or disorder, which disease or disorder is responsive to the inhibition of monoamine neurotransmitter re-uptake, which method comprises administration of effective amounts of a monoamine neurotransmitter re-uptake inhibitor comprising a 2,3-disubstituted tropane moiety, or a tautomer, a pharmaceutically acceptable salt, solvate, or physiologically functional derivative thereof, and at least one NMDA receptor antagonist or a pharmaceutically acceptable salt, solvate, or physiologically functional derivative thereof to a patient in need thereof in a combined form, or separately, or separately and sequentially, wherein the sequential administration is close in time or remote in time.

Diseases and/or disorders that may be prevented or treated by the present invention include: depression, dementia, pseudodementia, presenile dementia, senile dementia, dementia of Alzheimer-type, fronto-temporal dementia, HIV-related dementia, multi-infarct dementia, cerebrovascular dementia, Alzheimer's Disease, Lewy Body disease, Down syndrome, Pick's disease, Parkinson's Disease, memory deficits, attention deficits, cognitive dysfunction, memory dysfunction, age associated memory impairment, mild cognitive impairment, ageing-associated cognitive decline, age-related cognitive decline, multiple system atrophy, and neurodegenerative disorder with an associated cognitive deficit.

### DETAILED DESCRIPTION OF THE INVENTION

As a rule the monoamine neurotransmitter re-uptake inhibitor comprising a 2,3-disubstituted tropane moiety are compounds of the general formula (I) 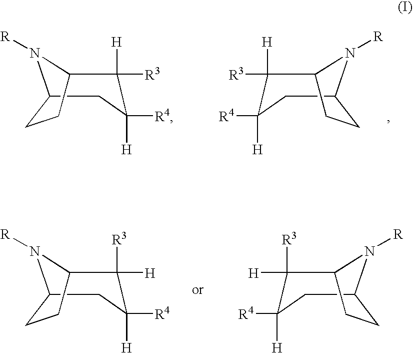
or a pharmaceutical acceptable addition salt thereof, or the N-oxide thereof, wherein
R is hydrogen, alkyl, alkenyl, alkynyl, cycloalkyl, cycloalkylalkyl or 2-hydroxyethyl;
R³ is
   CH₂—X—R′, wherein
      X is O, S, or NR″; wherein
         R″ is hydrogen or alkyl; and
      R′ is
         alkyl, alkenyl, alkynyl, cycloalkyl, cycloalkylalkyl, or —CO-alkyl;
         heteroaryl, which may be substituted one or more times with alkyl, cycloalkyl, or cycloalkylalkyl;
         phenyl, which may be substituted one or more times with substituents selected from the group consisting of halogen, CF₃, CN, alkoxy, alkyl, alkenyl, alkynyl, amino, nitro, and heteroaryl;
         phenylphenyl;
         pyridyl, which may be substituted one or more times with substituents selected from the group consisting of halogen, CF₃, CN, alkoxy, alkyl, alkenyl, alkynyl, amino, nitro, and heteroaryl;
         thienyl, which may be substituted one or more times with substituents selected from the group consisting of halogen, CF₃, CN, alkoxy, alkyl, alkenyl, alkynyl, amino, nitro, and heteroaryl; or
         benzyl, which may be substituted one or more times with substituents selected from the group consisting of halogen, CF₃, CN, alkoxy, alkyl, alkenyl, alkynyl, amino, nitro, and heteroaryl or
   (CH₂)ₙCO₂R¹¹, COR¹¹, or CH₂R¹², wherein
      R¹¹ is
         alkyl, cycloalkyl, or cycloalkylalkyl; phenyl, which may be substituted one or more times with substituents selected from the group consisting of halogen, CF₃, CN, alkoxy, alkyl, alkenyl, alkynyl, amino, nitro, and heteroaryl;
         phenylphenyl;
         pyridyl, which may be substituted one or more times with substituents selected from the group consisting of halogen, CF₃, CN, alkoxy, alkyl, alkenyl, alkynyl, amino, nitro, and heteroaryl;
         thienyl or O-thienyl, which may be substituted one or more times with substituents selected from the group consisting of halogen, CF₃, CN, alkoxy, alkyl alkenyl, alkynyl, amino, nitro, and heteroaryl; or
         benzyl;
      n is 0 or 1; and
      R¹² is
         O-phenyl, which may be substituted one or more times with substituents selected from the group consisting of halogen, CF₃, CN, alkoxy, alkyl, alkenyl, alkynyl, amino, nitro, and heteroaryl; or
         O—CO-phenyl, which may be substituted one or more times with substituents selected from the group consisting of halogen, CF₃, CN, alkoxy, alkyl, alkenyl, alkynyl, amino, nitro, and heteroaryl; or
   CH═NOR′, wherein
      R′ is
         hydrogen or O-hydrogen;
         alkyl, O-alkyl, cycloalkyl, cycloalkylalkyl, alkenyl, alkynyl or aryl, all of which may be substituted with —COOH;
         —COO-alkyl;
         —COO-cycloalkyl; or
         phenyl, which may be substituted one or more times with substituents selected from the group consisting of halogen, CF₃, CN, alkyl, cycloalkyl, alkoxy, cycloalkoxy, alkenyl, alkynyl, amino, and nitro;
R⁴ is
   3,4-methylenedioxyphenyl; or phenyl, benzyl, naphthyl, or heteroaryl, all of which may be substituted one or more times with substituents selected from the group consisting of halogen, CF₃, CN, alkoxy, cycloalkoxy, alkyl, cycloalkyl, alkenyl, alkynyl, amino, nitro, and heteroaryl.

In a special embodiment of the compound of general formula (I), R³ is
1,2,4-oxadiazol-3-yl, which may by substituted in the 5 position with
   alkyl, cycloalkyl, or cycloalkylalkyl;
   phenyl, which may be substituted one or more times with substituents selected from the group consisting of halogen, CF₃, CN, alkoxy, alkyl, alkenyl, alkynyl, amino, nitro, and heteroaryl; phenylphenyl; or benzyl, which may be substituted one or more times with substituents selected from the group consisting of halogen, CF₃, CN, alkoxy, alkyl, alkenyl, alkynyl, amino, nitro, and heteroaryl; or
1,2,4-oxadiazol-5-yl, which may by substituted in the 3 position with
   alkyl, cycloalkyl, or cycloalkylalkyl;
   phenyl, which may be substituted one or more times with substituents selected from the group consisting of halogen, CF₃, CN, alkoxy, alkyl, alkenyl, alkynyl, amino, nitro, and heteroaryl; phenylphenyl;
   benzyl, which may be substituted one or more times with substituents selected from the group consisting of halogen, CF₃, CN, alkoxy, alkyl, alkenyl, alkynyl, amino, nitro, and heteroaryl;
   pyridyl, which may be substituted one or more times with substituents selected from the group consisting of halogen, CF₃, CN, alkoxy, alkyl, alkenyl, alkynyl, amino, nitro and heteroaryl; or
   thienyl, which may be substituted one or more times with substituents selected from the group consisting of halogen, CF₃, CN, alkoxy, alkyl, alkenyl, alkynyl, amino, nitro and heteroaryl.

In a further special embodiment of the compound of general formula (I), R³ is
CH₂—X—R′, wherein
   X is
      O, S, or NR″; wherein
         R″ is hydrogen or alkyl; and
   R′ is alkyl, alkenyl, alkynyl, cycloalkyl, cycloalkylalkyl, or —CO-alkyl.

In a still further embodiment of the compound of general formula (I), R³ is
CH═NOR′, wherein
   R′ is
      hydrogen;
      alkyl, cycloalkyl, cycloalkylalkyl, alkenyl, alkynyl or aryl, all of which may be substituted with —COOH;
      —COO-alkyl;
      —COO-cycloalkyl; or
      phenyl, which may be substituted one or more times with substituents selected from the group consisting of halogen, CF₃, CN, alkyl, cycloalkyl, alkoxy, cycloalkoxy, alkenyl, alkynyl, amino, and nitro.

In a further special embodiment of the compound of general formula (I), R⁴ is phenyl, which is substituted once or twice with substituents selected from the group consisting of halogen, CF₃, CN, alkoxy, cycloalkoxy, alkyl, cycloalkyl, alkenyl, alkynyl, amino, nitro, and heteroaryl.

In a more special embodiment, R⁴ is phenyl substituted once or twice with chlorine.

In a further special embodiment, the tropane derivative having dopamine reuptake inhibitor activity is a (1R, 2R, 3S)-2,3-disubstituted tropane derivative of formula (I).

In a still further embodiment, the tropane derivative having dopamine reuptake inhibitory activity is a compound of general formula (I), wherein
R³ is
   —CH₂—X—R′, wherein X is O or S, and R′ is methyl, ethyl, propyl, or cyclopropylmethyl;
   —CH═NOR′, wherein R′ is hydrogen or alkyl, or
   1,2,4-oxadiazol-5-yl, which may by substituted in the 3 position with alkyl.

In a still further embodiment, the tropane derivative having dopamine reuptake inhibitory activity is a compound of general formula (I) wherein R is hydrogen, methyl, ethyl, or propyl.

In a still further embodiment, the tropane derivative having dopamine reuptake inhibitory activity is a compound of general formula I wherein R⁴ is 3,4-dichlorophenyl.

Preferably those monoamine neurotransmitter re-uptake inhibitor comprising a 2,3-disubstituted tropane moiety are compounds of formula (I1) 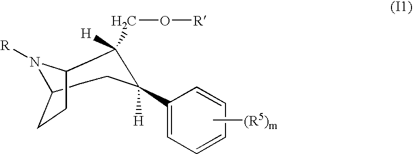
wherein
R represents a hydrogen atom or a C₁₋₆ alkyl group, preferably a hydrogen atom, a methyl or an ethyl group;
R⁵ each independently represents a halogen atom or a CF₃ or cyano group, preferably a fluorine, chlorine or bromine atom;
R represents a hydrogen atom or a C₁₋₆ alkyl or C₃₋₆-cycloalkyl-C₁₋₃-alkyl group, preferably a methyl, ethyl or n-propyl group; and
m is 0 or an integer from 1 to 3, preferably 1 or 2;
   or a tautomer, a pharmaceutically acceptable salt, solvate, or physiologically functional derivative thereof.

As used herein, the expression “C₁₋₆ alkyl” includes methyl and ethyl groups, and straight-chained and branched propyl, butyl, pentyl, and hexyl groups. Particular alkyl groups are methyl, ethyl, n-propyl, isopropyl, and t-butyl.

The expression “C₃₋₆ cycloalkyl,” as used herein, includes cyclic propyl, butyl, pentyl, and hexyl groups, such as cyclopropyl and cyclohexyl.

The term “halogen,” as used herein, includes fluorine, chlorine, bromine, and iodine, of which fluorine and chlorine are preferred.

The term “physiologically functional derivative,” as used herein, includes derivatives obtained from the compound of formula (I) under physiological conditions, these are, for example, N-oxides, which are formed under oxidative conditions.

The term “pharmaceutically acceptable acid addition salt,” as used herein, includes those salts that are selected from among the acid addition salts formed with hydrochloric acid, hydrobromic acid, sulphuric acid, phosphoric acid, methanesulphonic acid, acetic acid, fumaric acid, succinic acid, lactic acid, citric acid, tartaric acid, and maleic acid, the salts obtained from hydrochloric acid, hydrobromic acid, sulphuric acid, phosphoric acid, and acetic acid being particularly preferred. The salts of citric acid are of particular significance.

In a special embodiment, the tropane derivative having dopamine reuptake inhibitor activity is a compound of the general formula (I) selected from:
(1R,2R,3S)-2-(3-Cyclopropyl-1,2,4-oxadiazol-5-yl)-3-(4-fluorophenyl)-tropane;
(1R,2R,3S)-2-(3-Phenyl-1,2,4-oxadiazol-5-yl)-3-(4-fluorophenyl)-tropane;
(1R,2R,3S)-2-(3-Phenyl-1,2,4-oxadiazol-5-yl)-3-(4-methylphenyl)-tropane;
(1R,2R,3S)-2-(3-Benyl-1,2,4-oxadiazol-5-yl)-3-(4-fluorophenyl)-tropane;
(1R,2R,3S)-2-(3-(4-Phenyl-phenyl)-1,2,4-oxadiazol-5-yl)-3-(4-fluorophenyl)-tropane;
(1R,2R,3S)-2-(3-Phenyl-1,2,4-oxadiazol-5-yl)-3-(2-naphthyl)-tropane;
(1R,2R,3S)-3-(3,4-Dichlorophenyl)-tropane-2-aldoxime;
(1R,2R,3S)-3-(3,4-Dichlorophenyl)-tropane-2-O-methyl-aldoxime;
(1R,2R,3S)-3-(3,4-Dichlorophenyl)-tropane-2-O-benzyl-aldoxime;
(1R,2 R,3S)-3-(3,4-Dichlorophenyl)-tropane-2-O-ethoxycarbonylmethyl-aldoxime;
(1R,2R,3S)-3-(3,4-Dichlorophenyl)-tropane-2-O-methoxycarbonylmethyl-aldoxime;
(1R,2R,3S)-3-(3,4-Dichlorophenyl)-tropane-2-O-(1-ethoxycarbonyl-1,1-dimethyl-methyl)-aldoxime;
(1R,2R,3S)-3-(3,4-Dichlorophenyl)-tropane-2-O-carboxymethyl-2-aldoxime;
(1R,2R,3S)-N-Normethyl-3-(3,4-dichlorophenyl)-tropane-2-O-methyl-aldoxime;
(1R,2R,3S)-N-Normethyl-3-(3,4-dichlorophenyl)-tropane-2-O-benzyl-aldoxime;
(1R,2R,3S)-3-(4-Methylphenyl)-tropane-2-O-methyl-aldoxime;
(1R,2R,3S)-3-(3,4-Dichlorophenyl)-tropane-2-O-(1,1-dimethylethyl)-aldoxime;
(1R,2R,3S)-3-(4-Chlorophenyl)-tropane-2-O-aldoxime;
(1R,2R,3S)-3-(4-Chlorophenyl)-tropane-2-O-methylaldoximehydrochloride;
(1R,2R,3S)-3-(4-Chlorophenyl)-tropane-2-O-methoxycarbonylmethyl-aldoxime;
(1R,2R,3S)-3-(3,4-Dichlorophenyl)-tropane-2-O-(2-propynyl)-aldoxime;
(1R,2R,3S)-3-(3,4-Dichlorophenyl)-tropane-2-O-(2-methylpropyl)-aldoxime;
(1R,2R,3S)-3-(3,4-Dichlorophenyl)-tropane-2-O-cyclopropylmethyl-aldoxime;
(1R,2R,3S)-3-(3,4-Dichlorophenyl)-tropane-2-O-ethyl-aldoxime;
(1R,2R,3S)-2-Methoxymethyl-3-(3,4-dichlorophenyl)-tropane;
(1R,2R,3S)-2-Isopropoxymethyl-3-(3,4-dichlorophenyl)-tropane;
(1R,2R,3S)-2-Ethoxymethyl-3-(3,4-dichlorophenyl)-tropane;
(1R,2R,3S)-2-Ethoxymethyl-3-(3,4-dichlorophenyl)-nortropane;
(1R,2R,3S)-2-Cyclopropylmethyloxymethyl-3-(3,4-dichlorophenyl)-tropane;
(1R,2R,3S)-2-Methoxymethyl-3-(4-chlorophenyl)-tropane;
(1R,2R, 3S)-N-Normethyl-2-methoxymethyl-3-(4-chlorophenyl)-tropane;
(1R,2R,3S)-2-Ethoxymethyl-3-(4-chlorophenyl)-tropane;
(1R,2R,3S)-N-Normethyl-2-methoxymethyl-3-(3,4-dichlorophenyl)-tropane;
(1R,2R,3S)-N-Normethyl-2-ethoxymethyl-3-(3,4-dichlorophenyl)-tropane;
(1R,2R,3S)-N-Normethyl-2-ethoxymethyl-3-(4-chlorophenyl)-tropane;
(1R,2 R, 3S)-N-Normethyl-2-cyclopropylmethyloxymethyl-3-(4-chlorophenyl)-tropane;
(1R,2R,3S)-2-Cyclopropylmethyloxymethyl-3-(4-chlorophenyl)-tropane;
(1R,2R,3S)-2-Ethylthiomethyl-3-(3,4-dichlorophenyl)-tropane;
(1R,2R,3S)-2-Hydroxymethyl-3-(4-fluorophenyl)-tropane;
(1R,2R,3S)-2-Hydroxymethyl-3-(3,4-dichlorophenyl)-tropane;
(1R,2R, 3S)-N-Normethyl-N-(tert-butoxycarbonyl)-2-hydroxymethyl-3-(3,4-dichlorophenyl)-tropane;
(1R,2R,3S)-2-Hydroxymethyl-3-(4-chlorophenyl)-tropane;
(1R,2R,3S)-2-(3-(2-Furanyl)-1,2,4-oxadiazol-5-yl)-3-(3,4-dichlorophenyl)-tropane;
(1R,2R,3S)-2-(3-(3-Pyridyl)-1,2,4-oxadiazol-5-yl)-3-(3,4-dichlorophenyl)-tropane;
(1R,2R,3S)-N-Normethyl-N-allyl-2-(3-(4-pyridyl)-1,2,4-oxadiazol-5-yl)-3-(3,4-dichlorophenyl)-tropane;
(1R,2R,3S)-N-Normethyl-N-ethyl-2-(3-(4-pyridyl)-1,2,4-oxadiazol-5-yl)-3-(3,4-dichlorophenyl)-tropane;
(1R,2R,3S)-N-Normethyl-N-(2-hydroxyethyl)-2-(3-(4-pyridyl)-1,2,4-oxadiazol-5-yl)-3-(3,4-dichlorophenyl)-tropane;
(1R,2R, 3S)-N-Normethyl-2-(3-(4-pyridyl)-1,2,4-oxad iazol-5-yl)-3-(3,4-dichlorophenyl)-tropane;
(1R,2R,3S)-N-Normethyl-N-allyl-2-(3-(3-pyridyl)-1,2,4-oxadiazol-5-yl)-3-(3,4-dichlorophenyl)-tropane;
(1R,2R,3S)-N-Normethyl-N-allyl-2-(3-(2-pyridyl)-1,2,4-oxadiazol-5-yl)-3-(3,4-dichlorophenyl)-tropane;
(1R,2R,3S)-2-(3-(2-Thienyl)-1,2,4-oxadiazol-5-yl)-3-(4-chlorophenyl)-tropane;
(1R,2R,3S)-2-(3-(2-Thienyl)-1,2,4-oxadiazol-5-yl)-3-(3,4-dichlorophenyl)-tropane;
(1R,2R,3S)-2-(3-(4-Pyridyl)-1,2,4-oxadiazol-5-yl)-3-(3,4-dichlorophenyl)-tropane;
(1R,2R,3S)-2-(3-(2-Pyridyl)-1,2,4-oxadiazol-5-yl)-3-(3,4-dichlorophenyl)-tropane;
(1R,2 R, 3S)-2-(3-(4-Pyridyl)-1,2,4-oxadiazol-5-yl)-3-(4-chlorophenyl)-tropane;
(1R,2 R, 3S)-2-(3-(3-Pyridyl)-1,2,4-oxad iazol-5-yl)-3-(4-chlorophenyl)-tropane;
(1R,2R,3S)-2-(3-2-Pyridyl)-1,2,4-oxadiazol-5-yl)-3-(4-chlorophenyl)-tropane;
(1R,2R,3S)-2-(3-Phenyl-1,2,4-oxadiazol-5-yl)-3-(4-fluorophenyl)-tropane;
(1R,2R,3S)-2-(3-Phenyl-1,2,4-oxadiazol-5-yl)-3-(4-methylphenyl)-tropane;
(1R,2R,3S)-2-(3-Benzyl-1,2,4-oxadiazol-5-yl)-3-(4-fluorophenyl)-tropane;
(1R,2R,3S)-2-(3-(4-Phenylphenyl)-1,2,4-oxadiazol-5-yl)-3-(4-fluorophenyl)-tropane;
(1R,2R,3S)-2-(3-Phenyl-1,2,4-oxadiazol-5-yl)-3-(2-naphthyl)-tropane;
(1R,2R,3S)-2-(4-Chlorophenoxy-methyl)-3-(4-fluorophenyl)-tropane;
(1R,2R,3S)-2-(4-Chlorophenoxy-methyl)-3-(4-fluorophenyl)-tropane;
(1R,2R,3S)-2-(4-Chlorophenoxy-methyl)-3-(3,4-dichlorophenyl)-tropane;
(1R,2R,3S)-2-(4-Chlorophenoxy-methyl)-3-(4-methylphenyl)-tropane;
(1R,2R,3S)-2-(4-Benzoyloxy-methyl)-3-(4-fluorophenyl)-tropane;
(1R,2R,3S)-2-Carbomethoxy-3-(2-naphthyl)-tropane;
(1R,2R,3S)-2-Carbomethoxy-3-(3,4-dichlorophenyl)-tropane;
(1R,2R, 3S)-2-Carbomethoxy-3-benzyl-tropane;
(1R,2R,3S)-2-Carbomethoxy-3-(4-chlorophenyl)-tropane;
(1R,2R,3S)-2-Carbomethoxy-3-(4-methylphenyl)-tropane;
(1R,2R,3S)-2-Carbomethoxy-3-(1-naphthyl)-tropane;
(1R,2R,3S)-2-Carbomethoxy-3-(4-phenylphenyl)-tropane;
(1R,2R,3S)-2-Carbomethoxy-3-(4-t-butyl-phenyl)-tropane;
(1R,2R,3S)-2-(4-Fluoro-benzoyl)-3-(4-fluorophenyl)-tropane; or
   a pharmaceutically acceptable addition salt thereof.

Most preferred is the compound of formula (IA) 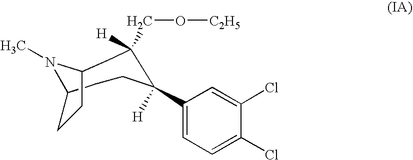
or a pharmaceutically acceptable salt thereof, in particular the citrate thereof.

NMDA receptor antagonists that may be used include any that are known to the skilled person and those which will become available in the future. Examples are aptiganel, budipine, eliprodil, felbamate gacyclidine, remacemide, lanicemine, memantine, midafotel, remacemide, selfotel, and sinnabidol.

Most preferred is a combination of the compound of formula (IA) with memantine, which is 3,5-dimethyl-1-adamantanamine of formula (II), 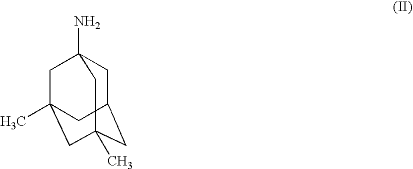
in particular in form of its hydrochloride or sulfate.

The pharmaceutical compositions of the present invention are suitable for oral, intravenous, intravascular, intraperitoneal, subcutaneous, intramuscular, inhalative, topical, patch, or suppository administration.

The pharmaceutical compositions of the present invention are preferably in unit dosage forms, such as tablets, pills, capsules, powders, granules, sterile parenteral solutions, or suspensions, metered aerosol or liquid sprays, drops, ampoules, transdermal patches, auto-injector devices, or suppositories; for oral, parenteral, intranasal, sublingual, or rectal administration, or for administration by inhalation or insufflation. For preparing solid compositions such as tablets, the principal active ingredient is mixed with a pharmaceutical carrier, e.g., conventional tableting ingredients, such as corn starch, cellulose, carboxymethylcellulose, hydroxypropylmethylcellulose, lactose, sucrose, sorbitol, talc, silicon dioxide, polyethylene glycol, stearic acid, magnesium stearate, and dicalcium phosphate, or gums, or surfactants, such as sorbitan monooleate, polyethylene glycol, and other pharmaceutical diluents, e.g., water, to form a solid pre-formulation composition containing a homogeneous mixture of a compound of the present invention, or a pharmaceutically acceptable salt thereof. When referring to these pre-formulation compositions as homogeneous, it is meant that the active ingredient is dispersed evenly throughout the composition so that the composition may be readily subdivided into equally effective unit dosage forms, such as tablets, pills, and capsules.

This solid pre-formulation composition is then subdivided into unit dosage forms of the type described above containing from 0.05 to 10,000 mg, in particular 0.1 to about 500 mg, most preferably 0.1 to 250 mg, of each active ingredient of the present invention. Typical unit dosage forms contain from 0.1 to 100 mg, for example 0.1, 0.5, 1, 2, 5, 10, 25, 50, or 100 mg, of each active ingredient.

The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Similarly, cachets and lozenges are included. Tablets, powders, capsules, pills, cachets, and lozenges can be used as solid forms suitable for oral administration.

The liquid forms in which the novel compositions of the present invention may be incorporated for administration orally or by injection include aqueous solutions, suitably flavoured syrups, aqueous or oil suspensions, and flavoured emulsions with edible oils, such as cottonseed oil, sesame oil, coconut oil, or peanut oil, as well as elixirs and similar pharmaceutical vehicles. Suitable dispersing or suspending agents for aqueous suspensions include synthetic and natural gums, such as tragacanth, acacia, alginate, dextran, sodium carboxymethylcellulose, methylcellulose, polyvinyl-pyrrolidone, or gelatin.

For preparing suppositories, a low melting wax, such as admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams, or sprays containing, in addition to the active ingredient, such carriers as are known in the art to be appropriate.

Administration to the respiratory tract may also be achieved by means of an aerosol formulation in which the active ingredient is provided in a pressurised pack with a suitable propellant such as a chlorofluorocarbon (CFC) or fluorohydrocarbon (HFC), for example, dichlorodifluoromethane, trichlorofluoromethane, dichlorotetrafluoroethane, 1,1,1,2-tetrafluoroethan (HFC-134(a)), or 1,1,1,2,3,3,3-heptafluoroprpane, carbon dioxide, or other suitable gas. The aerosol may conveniently also contain a surfactant such as lecithin and/or a co-solvent such as ethanol. The dose of drug may be controlled by provision of a metered valve.

Alternatively, the active ingredients may be provided in the form of a dry powder, for example, a powder mix of the compound in a suitable powder base, such as lactose, starch, and starch derivatives, such as hydroxypropylmethyl cellulose and polyvinylpyrrolidone (PVP). Conveniently, the powder carrier will form a gel in the nasal cavity. The powder composition may be presented in unit dose form, for example, in capsules or cartridges of, e.g., gelatin, or blister packs from which the powder may be administered by means of an inhaler.

In formulations intended for administration to the respiratory tract, including intranasal formulations, the compound will generally have a small particle size, for example, of the order of 5 microns or less. Such a particle size may be obtained by means known in the art, for example by micronization.

For the treatment of a neurodegenerative condition, a suitable dosage level is about 0.01 to 250 mg/kg per day, preferably about 0.01 to 100 mg/kg per day, and especially about 0.01 to 5 mg/kg of body weight per day of each active ingredient. The compounds may be administered on a regimen of 1 to 4 times per day. In some cases, however, dosage outside these limits may be used.

Most preferably the composition of the invention will be used for the treatment or prevention of one or more of the following neurodegenerative conditions: pseudodementia, dementia, including dementia of Alzheimer-type, Alzheimer's Disease, presenile dementia, senile dementia, Lewy-Body dementia, Down syndrome, fronto-temporal dementia, HIV-related dementia, Pick's Disease, Parkinson's Disease, cerebrovascular dementia, multi-infarct dementia, memory deficits, attention deficits, cognitive dysfunction, memory dysfunction, mild cognitive impairment (MCI), age-associated memory impairment (AAMI), ageing-associated cognitive decline, age-related cognitive decline, ALS, and multiple system atrophy.

Preferably the weight ratio of the monoamine neurotransmitter re-uptake inhibitor to the NMDR receptors antagonist ranges from 50:1 to 1:300, in particular from 1:1 to 1:200, most preferably from 1:2 to 1:100.

Most preferred are the following daily dose rates: 0.5-20 mg, preferably 1.0-10 mg of memantine and 0.01-2.0 mg of the compound of formula (IA);

The Examples that follow serve to illustrate some formulations according to the invention. They are intended solely as possible procedures described by way of example, without restricting the invention to their content.

### EXAMPLE 1

### Film-Coated Tablet

| | |
|---|---|
| Constituents | mg/tablet |

Core

| | |
|---|---|
| (IA) citrate | 0.793 |
| Memantine hydrochloride | 5.988 |
| Lactose monohydrate (200 mesh) | 98.125 |
| Microcrystalline cellulose (grade PH 101) | 63.000 |
| Corn starch | 6.300 |
| Purified water* | (q.s.)* |
| Sodiumstarchglycolate | 3.600 |
| Colloidal silicon dioxide | 0.900 |
| Magnesium stearate | 1.800 |

Coating

| | |
|---|---|
| Hydroxyproylmethylcellulose 2910 | 2.750 |
| Polyethylene Glycol 400 | 0.325 |
| Titanium dioxide | 1.000 |
| Talc | 0.925 |
| Purified water* | (q.s.)* |
| Total weight film coated tablet | 185.000 |

*does not appear in final product

### EXAMPLE 2

### Bilayer Tablet

Mg/tablet

1^{st} tablet layer Constituents

| | |
|---|---|
| (IA) citrate | 0.396 |
| Lactose monohydrate (200 mesh) | 70.104 |
| Microcrystalline cellulose (grade PH 101) | 42.000 |
| Corn starch | 4.200 |
| Purified water | (q.s.)* |
| Sodiumstarchglycolate | 2.400 |
| Magnesium stearate | 0.900 |

2^{nd} tablet layer Constituents

| | |
|---|---|
| Memantine hydrochloride | 4.791 |
| Sorbitol, powder | 116.322 |
| Microcrystalline Cellulose | 14.000 |
| Crospovidone | 2.800 |
| Magnesium stearate | 1.750 |
| Total weight bilayer tablet | 260.000 |

*does not appear in final product

The advantageous effect of the combination of the present invention can be shown, for example, by comparing the combined dosage of the combination with dosages of the same amount of each of the active ingredients separately on subjects using the Mini-Mental State Examination (MMSE), as described in Folstein and Folstein, J. Psychiat. Res., 1975, 12,189-198, or a variant thereof, as discussed in Tombaugh and Mcintyre, JAGS, 1992, 40, 922-935.

1. A composition comprising:
a 2,3-disubstituted tropane moiety, or a tautomer, pharmaceutically acceptable salt, solvate, or physiologically functional derivative thereof,
at least one N-methyl-D-aspartate receptors antagonist, or a pharmaceutically acceptable salt, solvate, or physiologically functional derivative thereof.

2. A composition according to claim 1, wherein the 2,3-disubstituted tropane moiety is a compound of formula (I) 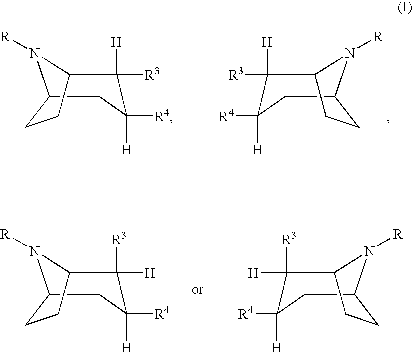
or an addition salt or N-oxide thereof, wherein
R is hydrogen, alkyl, alkenyl, alkynyl, cycloalkyl, cycloalkylalkyl, or 2-hydroxyethyl;
R³ is
CH₂—X—R′, wherein
X is O, S, or NR”, wherein
R″ is hydrogen or alkyl; and
R′ is
alkyl, alkenyl, alkynyl, cycloalkyl, cycloalkylalkyl, or —CO— alkyl;
heteroaryl, which may be substituted one or more times with alkyl, cycloalkyl, or cycloalkylalkyl;
phenyl, which may be substituted one or more times with substituents selected from the group consisting of halogen, CF₃, CN, alkoxy, alkyl, alkenyl,alkynyl, amino,
nitro, and heteroaryl;
phenylphenyl;
pyridyl, which may be substituted one or more times with substituents selected from the group consisting of halogen, CF₃, CN, alkoxy, alkyl, alkenyl, alkynyl, amino, nitro, and heteroaryl;
thienyl, which may be substituted one or more times with substituents selected from the group consisting of halogen, CF₃, CN, alkoxy, alkyl, alkenyl, alkynyl, amino, nitro, and heteroaryl; or
benzyl, which may be substituted one or more times with substituents selected from the group consisting of halogen, CF₃, CN, alkoxy, alkyl, alkenyl, alkynyl, amino, nitro, and heteroaryl; or
(CH₂)ₙCO₂R¹¹, COR¹¹, or CH₂R¹² wherein
R¹¹ is
alkyl, cycloalkyl, or cycloalkylalkyl;
phenyl, which may be substituted one or more times with substituents selected from the group consisting of halogen, CF₃, CN, alkoxy, alkyl, alkenyl, alkynyl, amino, nitro, and heteroaryl;
phenylphenyl;
pyridyl, which may be substituted one or more times with substituents selected from the group consisting of halogen, CF₃, CN, alkoxy, alkyl, alkenyl, alkynyl, amino, nitro, and heteroaryl;
thienyl or O-thienyl, which may be substituted one or more times with substituents selected from the group consisting of halogen, CF₃, CN, alkoxy, alkyl, alkenyl, alkynyl, amino, nitro, and heteroaryl; or
benzyl;
n is 0 or 1; and
R¹² is
O-phenyl, which may be substituted one or more times with substituents selected from the group consisting of halogen, CF₃, CN, alkoxy, alkyl, alkenyl, alkynyl, amino, nitro, and heteroaryl; or
O—CO-phenyl, which may be substituted one or more times with substituents selected from the group consisting of halogen, CF₃, CN, alkoxy, alkyl, alkenyl, alkynyl, amino, nitro, and heteroaryl; or
CH═NOR′ wherein
R′ is
hydrogen or O-hydrogen;
alkyl, O-alkyl, cycloalkyl, cycloalkylalkyl, alkenyl, alkynyl or aryl, all of which may be substituted with —COOH;
—COO-alkyl;
—COO-cycloalkyl; or
phenyl, which may be substituted one or more times with substituents selected from the group consisting of halogen, CF₃, CN, alkyl, cycloalkyl, alkoxy, cycloalkoxy, alkenyl, alkynyl, amino, and nitro; and
R⁴ is
3,4-methylenedioxyphenyl; or
phenyl, benzyl, naphthyl, or heteroaryl, all of which may be substituted one or more times with substituents selected from the group consisting of halogen, CF3, CN, alkoxy, cycloalkoxy, alkyl, cycloalkyl, alkenyl, alkynyl, amino, nitro, and heteroaryl.

3. A composition according to claim 2, wherein R⁴ is phenyl, which is substituted once or twice with substituents selected from the group consisting of: halogen, CF₃, CN, alkoxy, cycloalkoxy, alkyl, cycloalkyl, alkenyl, alkynyl, amino, nitro, and heteroaryl.

4. A composition according to claim 2, wherein R⁴ is phenyl, which is substituted once or twice with chlorine.

5. A composition according to claim 1, wherein the 2,3-disubstituted tropane moiety is a compound of formula (I) 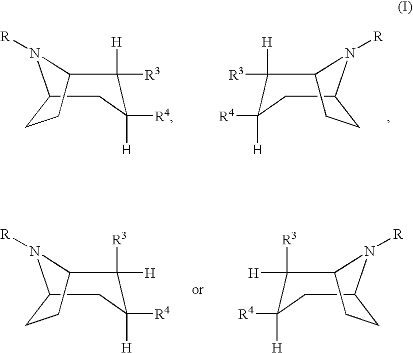
or an addition salt or N-oxide thereof, wherein
R is hydrogen, alkyl, alkenyl, alkynyl, cycloalkyl, cycloalkylalkyl, or 2-hydroxyethyl;
R³ is
CH₂—X—R′, wherein
X is O, S, or NR″, wherein
R″ is hydrogen or alkyl; and
R′ is alkyl, alkenyl, alkynyl, cycloalkyl, cycloalkylalkyl, or —CO-alkyl; and
R⁴ is
3,4-methylenedioxyphenyl; or
phenyl, benzyl, naphthyl, or heteroaryl, all of which may be substituted one or more times with substituents selected from the group consisting of halogen, CF3, CN, alkoxy, cycloalkoxy, alkyl, cycloalkyl, alkenyl, alkynyl, amino, nitro, and heteroaryl.

6. A composition according to claim 1, wherein the 2,3-disubstituted tropane moiety is a compound of formula (I) 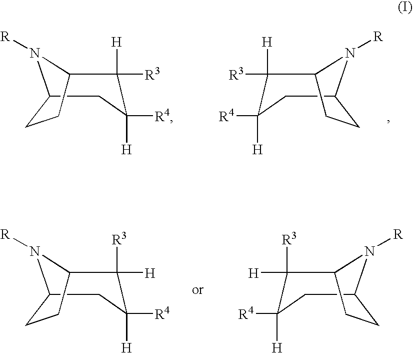
or an addition salt or N-oxide thereof, wherein
R is hydrogen, alkyl, alkenyl, alkynyl, cycloalkyl, cycloalkylalkyl, or 2-hydroxyethyl;
R³ is
CH═NOR′ wherein
R′ is
hydrogen;
alkyl, cycloalkyl, cycloalkylalkyl, alkenyl, alkynyl or aryl, all of which may be substituted with —COOH;
—COO-alkyl;
—COO-cycloalkyl; or
phenyl, which may be substituted one or more times with substituents selected from the group consisting of halogen, CF₃, CN, alkyl, cycloalkyl, alkoxy, cycloalkoxy, alkenyl, alkynyl, amino, and nitro; and
R⁴ is
3,4-methylenedioxyphenyl; or
phenyl, benzyl, naphthyl, or heteroaryl, all of which may be substituted one or more times with substituents selected from the group consisting of halogen, CF3, CN, alkoxy, cycloalkoxy, alkyl, cycloalkyl, alkenyl, alkynyl, amino, nitro, and heteroaryl.

7. A composition according to claim 1, wherein the 2,3-disubstituted tropane moiety is a compound of formula (I1) 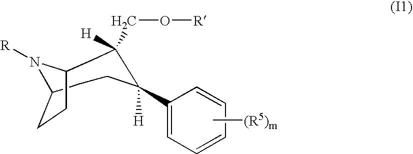
wherein
R represents a hydrogen atom or a C₁₋₆ alkyl group;
R⁵ represents a halogen atom or a CF₃ or cyano group;
R′ represents a hydrogen atom or a C₁₋₆ alkyl, or C₃₋₆-cycloalkyl-C₁₋₃-alkyl group; and
m is 0 or an integer from 1 to 3;
or a tautomer, pharmaceutically acceptable salt, solvate, or physiologically functional derivative thereof.

8. A composition according to claim 7, wherein:
R represents hydrogen, or a methyl or ethyl group;
R⁵ represents fluorine, chlorine, or bromine;
R′ represents a methyl, ethyl, or n-propyl group; and
m is 1 or 2.

9. A composition according to claim 1, wherein the 2,3-disubstituted tropane moiety is selected from the group consisting of:
(1R,2R,3S)-2-(3-Cyclopropyl-1,2,4-oxadiazol-5-yl)-3-(4-fluorophenyl)-tropane;
(1R,2R,3S)-2-(3-Phenyl-1,2,4-oxadiazol-5-yl)-3-(4-fluorophenyl)-tropane;
(1R,2R,3S)-2-(3-Phenyl-1,2,4-oxadiazol-5-yl)-3-(4-methylphenyl)-tropane;
(1R,2R,3S)-2-(3-Benyl-1,2,4-oxadiazol-5-yl)-3-(4-fluorophenyl)-tropane;
(1R,2R,3S)-2-(3-(4-Phenyl-phenyl)-1,2,4-oxadiazol-5-yl)-3-(4-fluorophenyl)-tropane;
(1R,2R,3S)-2-(3-Phenyl-1,2,4-oxadiazol-5-yl)-3-(2-naphthyl)-tropane;
(1R,2R,3S)-3-(3,4-Dichlorophenyl)-tropane-2-aldoxime;
(1R,2R,3S)-3-(3,4-Dichlorophenyl)-tropane-2-O-methyl-aldoxime;
(1R,2 R, 3S)-3-(3,4-Dichlorophenyl)-tropane-2-O-benzyl-aldoxime;
(1R,2R,3S)-3-(3,4-Dichlorophenyl)-tropane-2-O-ethoxycarbonylmethyl-aldoxime;
(1R,2R,3S)-3-(3,4-Dichlorophenyl)-tropane-2-O-methoxycarbonylmethyl-aldoxime;
(1R,2 R, 3S)-3-(3,4-Dichlorophenyl)-tropane-2-O-(1-ethoxycarbonyl-1,1-dimethyl-methyl)-aldoxime;
(1R,2R,3S)-3-(3,4-Dichlorophenyl)-tropane-2-O-carboxymethyl-2-aldoxime;
(1R,2R,3S)-N-Normethyl-3-(3,4-dichlorophenyl)-tropane-2-O-methyl-aldoxime;
(1R,2R,3S)-N-Normethyl-3-(3,4-dichlorophenyl)-tropane-2-O-benzyl-aldoxime;
(1R,2R,3S)-3-(4-Methylphenyl)-tropane-2-O-methyl-aldoxime;
(1R,2R,3S)-3-(3,4-Dichlorophenyl)-tropane-2-O-(1,1-dimethylethyl)-aldoxime;
(1R,2R,3S)-3-(4-Chlorophenyl)-tropane-2-O-aldoxime;
(1R,2R,3S)-3-(4-Chlorophenyl)-tropane-2-O-methylaldoxime hydrochloride;
(1R,2R,3S)-3-(4-Chlorophenyl)-tropane-2-O-methoxycarbonylmethyl-aldoxime;
(1R,2R,3S)-3-(3,4-Dichlorophenyl)-tropane-2-O-(2-propynyl)-aldoxime;
(1R,2R,3S)-3-(3,4-Dichlorophenyl)-tropane-2-O-(2-methylpropyl)-aldoxime;
(1R,2R,3S)-3-(3,4-Dichlorophenyl)-tropane-2-O-cyclopropylmethyl-aldoxime;
(1R,2R,3S)-3-(3,4-Dichlorophenyl)-tropane-2-O-ethyl-aldoxime;
(1R,2R,3S)-2-Methoxymethyl-3-(3,4-dichlorophenyl)-tropane;
(1R,2R,3S)-2-Isopropoxymethyl-3-(3,4-dichlorophenyl)-tropane;
(1R,2R,3S)-2-Ethoxymethyl-3-(3,4-dichlorophenyl)-tropane;
(1R,2R,3S)-2-Ethoxymethyl-3-(3,4-dichlorophenyl)-nortropane;
(1R,2R,3S)-2-Cyclopropylmethyloxymethyl-3-(3,4-dichlorophenyl)-tropane;
(1R,2R,3S)-2-Methoxymethyl-3-(4-chlorophenyl)-tropane;
(1R,2R,3S)-N-Normethyl-2-methoxymethyl-3-(4-chlorophenyl)-tropane;
(1R,2R,3S)-2-Ethoxymethyl-3-(4-chlorophenyl)-tropane;
(1R,2R,3S)-N-Normethyl-2-methoxymethyl-3-(3,4-dichlorophenyl)-tropane;
(1 R,2R,3S)-N-Normethyl-2-ethoxymethyl-3-(3,4-dichlorophenyl)-tropane;
(1R,2R,3S)-N-Normethyl-2-ethoxymethyl-3-(4-chlorophenyl)-tropane;
(1R,2R,3S)-N-Normethyl-2-cyclopropylmethyloxymethyl-3-(4-chlorophenyl)-tropane;
(1R, 2 R, 3S)-2-Cyclopropylmethyloxymethyl-3-(4-chlorophenyl)-tropane;
(1R,2R,3S)-2-Ethylthiomethyl-3-(3,4-dichlorophenyl)-tropane;
(1R,2R,3S)-2-Hydroxymethyl-3-(4-fluorophenyl)-tropane;
(1R,2R,3S)-2-Hydroxymethyl-3-(3,4-dichlorophenyl)-tropane;
(1R,2R,3S)-N-Normethyl-N-(tert-butoxycarbonyl)-2-hydroxymethyl-3-(3,4-dichlorophenyl)-tropane;
(1R,2R,3S)-2-Hydroxymethyl-3-(4-chlorophenyl)-tropane;
(1R,2R,3S)-2-(3-(2-Furanyl)-1,2,4-oxadiazol-5-yl)-3-(3,4-dichlorophenyl)-tropane;
(1R,2R,3S)-2-(3-(3-Pyridyl)-1,2,4-oxadiazol-5-yl)-3-(3,4-dichlorophenyl)-tropane;
(1R,2 R, 3S)-N-Normethyl-N-allyl-2-(3-(4-pyridyl)-1,2,4-oxad iazol-5-yl)-3-(3,4-dichlorophenyl)-tropane;
(1R,2R,3S)-N-Normethyl-N-ethyl-2-(3-(4-pyridyl)-1,2,4-oxadiazol-5-yl)-3-(3,4-dichlorophenyl)-tropane;
(1R,2R,3S)-N-Normethyl-N-(2-hydroxyethyl)-2-(3-(4-pyridyl)-1,2,4-oxadiazol-5-yl)-3-(3,4-dichlorophenyl)-tropane;
(1R,2R,3S)-N-Normethyl-2-(3-(4-pyridyl)-1,2,4-oxadiazol-5-yl)-3-(3,4-dichlorophenyl)-tropane;
(1R,2R,3S)-N-Normethyl-N-allyl-2-(3-(3-pyridyl)-1,2,4-oxadiazol-5-yl)-3-(3,4-dichlorophenyl)-tropane;
(1R, 2 R, 3S)-N-Normethyl-N-allyl-2-(3-(2-pyridyl)-1,2,4-oxad iazol-5-yl)-3-(3,4-dichlorophenyl)-tropane;
(1R,2R,3S)-2-(3-(2-Thienyl)-1,2,4-oxadiazol-5-yl)-3-(4-chlorophenyl)-tropane;
(1R,2R,3S)-2-(3-(2-Thienyl)-1,2,4-oxadiazol-5-yl)-3-(3,4-dichlorophenyl)-tropane;
(1R,2R,3S)-2-(3-(4-Pyridyl)-1,2,4-oxadiazol-5-yl)-3-(3,4-dichlorophenyl)-tropane;
(1R,2 R, 3S)-2-(3-(2-Pyridyl)-1,2,4-oxad iazol-5-yl)-3-(3,4-d ichlorophenyl)-tropane;
(1R,2 R, 3S)-2-(3-(4-Pyridyl)-1,2,4-oxadiazol-5-yl)-3-(4-chlorophenyl)-tropane;
(1R,2R,3S)-2-(3-(3-Pyridyl)-1,2,4-oxadiazol-5-yl)-3-(4-chlorophenyl)-tropane;
(1R,2R,3S)-2-(3-2-Pyridyl)-1,2,4-oxadiazol-5-yl)-3-(4-chlorophenyl)-tropane;
(1R,2R,3S)-2-(3-Phenyl-1,2,4-oxadiazol-5-yl)-3-(4-fluorophenyl)-tropane;
(1R,2R,3S)-2-(3-Phenyl-1,2,4-oxadiazol-5-yl)-3-(4-methylphenyl)-tropane;
(1R,2R,3S)-2-(3-Benzyl-1,2,4-oxadiazol-5-yl)-3-(4-fluorophenyl)-tropane;
(1R,2R,3S)-2-(3-(4-Phenylphenyl)-1,2,4-oxadiazol-5-yl)-3-(4-fluorophenyl)-tropane;
(1R,2R,3S)-2-(3-Phenyl-1,2,4-oxadiazol-5-yl)-3-(2-naphthyl)-tropane;
(1R,2R,3S)-2-(4-Chlorophenoxy-methyl)-3-(4-fluorophenyl)-tropane;
(1R,2R,3S)-2-(4-Chlorophenoxy-methyl)-3-(4-fluorophenyl)-tropane;
(1R,2R,3S)-2-(4-Chlorophenoxy-methyl)-3-(3,4-dichlorophenyl)-tropane;
(1R,2R,3S)-2-(4-Chlorophenoxy-methyl)-3-(4-methylphenyl)-tropane;
(1R,2R,3S)-2-(4-Benzoyloxy-methyl)-3-(4-fluorophenyl)-tropane;
(1R,2R,3S)-2-Carbomethoxy-3-(2-naphthyl)-tropane;
(1R,2R,3S)-2-Carbomethoxy-3-(3,4-dichlorophenyl)-tropane;
(1R,2R,3S)-2-Carbomethoxy-3-benzyl-tropane;
(1R,2R,3S)-2-Carbomethoxy-3-(4-chlorophenyl)-tropane;
(1R,2R,3S)-2-Carbomethoxy-3-(4-methylphenyl)-tropane;
(1R,2R,3S)-2-Carbomethoxy-3-(1-naphthyl)-tropane;
(1R,2R,3S)-2-Carbomethoxy-3-(4-phenylphenyl)-tropane;
(1R,2 R, 3S)-2-Carbomethoxy-3-(4-t-butyl-phenyl)-tropane; and
(1R,2R,3S)-2-(4-Fluoro-benzoyl)-3-(4-fluorophenyl)-tropane,
or a pharmaceutically acceptable addition salt of such 2,3-disubstituted tropane moiety.

10. A composition according to claim 1, wherein the 2,3-disubstituted tropane moiety is a compound of formula (IA) 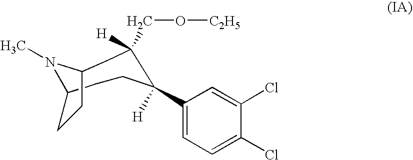
or a pharmaceutically acceptable salt thereof;

11. A composition according to claim 1, wherein the N-methyl-D-aspartate receptors antagonist is selected from the group consisting of: aptiganel, budipine, eliprodil, felbamate gacyclidine, remacemide, lanicemine, memantine, midafotel, remacemide, selfotel, and sinnabidol, and mixtures thereof.

12. A composition according to claim 1 that is suitable for oral, intravenous, intravascular, intraperitoneal, subcutaneous, intramuscular, inhalative, topical, patch, or suppository administration.

13. A composition according to claim 1, wherein the 2,3-disubstituted tropane moiety and the N-methyl-D-aspartate receptors antagonist are each present in a weight of about 0.05 mg to about 10,000 mg.

14. A composition according to claim 1, wherein the weight ratio of the 2,3-disubstituted tropane moiety to the N-methyl-D-aspartate receptors antagonist is about 50:1 to about 1:300.

15. A method for the prevention or treatment of a disease or disorder that is responsive to a monoamine neurotransmitter re-uptake inhibitor, a N-methyl-D-aspartate receptors antagonist, or both, the method comprising jointly, separately, or sequentially administering, to a patient in need thereof, effective amounts of: (i) a 2,3-disubstituted tropane moiety, or a tautomer, pharmaceutically acceptable salt, solvate, or physiologically functional derivative thereof, and (ii) an N-methyl-D-aspartate receptors antagonist, or a pharmaceutically acceptable salt, solvate, or physiologically functional derivative thereof.

16. A method according to claim 15, wherein said disease or disorder is selected from the group consisting of: pseudodementia, dementia, Alzheimer-type dementia, Alzheimer's Disease, presenile dementia, senile dementia, Lewy-Body dementia, Down syndrome, fronto-temporal dementia, HIV-related dementia, Pick's Disease, Parkinson's Disease, cerebrovascular dementia, multi-infarct dementia, memory deficits, attention deficits, cognitive dysfunction, memory dysfunction, mild cognitive impairment (MCI), age-associated memory impairment (AAMI), ageing-associated cognitive decline, age-related cognitive decline, and multiple system atrophy.

17. A method according to claim 15, wherein the effective amounts of the 2,3-disubstituted tropane moiety and the N-methyl-D-aspartate receptors antagonist are each about 0.5 mg to about 20 mg per day.

18. A method according to claim 15, wherein the weight ratio of the effective amount of the 2,3-disubstituted tropane moiety to the effective amount of the N-methyl-D-aspartate receptors antagonist is about 50:1 to about 1:300.

19. A pharmaceutical kit comprising comprising:
a first dosage form comprising a monoamine neurotransmitter re-uptake inhibitor comprising a 2,3-disubstituted tropane moiety, or a tautomer, pharmaceutically acceptable salt, solvate, or physiologically functional derivative thereof; and
a second dosage form comprising at least one N-methyl-D-aspartate receptors antagonist, or a pharmaceutically acceptable salt, solvate, or physiologically functional derivative thereof.

20. A pharmaceutical kit according to claim 19, wherein the first dosage form comprises a compound of formula (IA): 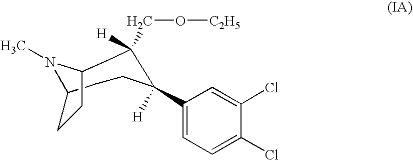
and the second dosage form comprises a compound selected from the group consisting of: aptiganel, budipine, eliprodil, felbamate gacyclidine, remacemide, lanicemine, memantine, midafotel, remacemide, selfotel, and sinnabidol, and mixtures thereof.
